(19) United States
(12) Plant Patent Application Publication
(10) Pub. No.: US 2019/0357414 P1
(43) Pub. Date: Nov. 21, 2019

(54) Stokesia Plant Named 'Mega Mels'

(50) ;

(71) Applicant: Van Heesbeen Cultures B.V., Rijsbergen (NL)

(72) Inventor: Ad Van Heesbeen, Rijsbergen (NL)

(73) Assignee: Van Heesbeen Cultures B.V., Rijsbergen (NL)

(21) Appl. No.: 16/501,670

(22) Filed: May 20, 2019

(30) Foreign Application Priority Data
May 18, 2018 (QZ) 2018/1335

(51) Int. Cl. A01H 6/14

(52) U.S. Cl. PLT/484

(57) ABSTRACT

A new and distinct Stokesia plant named ‘Mega Mels’ which is characterized by the combination of a large plant size, globular habit, large light purple flowers with yellow centers, and the stability of these characteristics from generation to generation.

### CROSS REFERENCE TO RELATED APPLICATIONS

This application claims priority to the Community Plant Variety Rights application number 2018/1335, filed May 18, 2018, which is herein incorporated by reference.

Latin name of the genus and species: The Latin name of the genus and species of the novel variety disclosed herein is Stokesia laevis.

Variety denomination: The inventive cultivar of Stokesia disclosed herein has been given the variety denomination ‘Mega Mels’.

### BACKGROUND OF THE INVENTION

Parentage: ‘Mega Mels’ is a seedling selection resulting from the controlled pollination of the seed parent, Stokesia laevis ‘Mels Blue’ (U.S. Plant Pat. No. 23,090), with an unnamed Stokesia laevis plant (not patented), the pollen parent. The crossing was made by the inventor in April of 2014 at a commercial greenhouse in Rijsbergen, the Netherlands. In August of 2015, one seedling from said cross was observed which exhibited large, light purple flowers. The seedling was isolated for further evaluation in order to confirm the distinctness and stability of the characteristics first observed. Upon confirmation of distinctness and stability, ‘Mega Mels’ was selected for commercialization.

Asexual Reproduction: Asexual reproduction of ‘Mega Mels’, by way of softwood stem cuttings, was first performed in August of 2015 in Rijsbergen, the Netherlands. Through five subsequent generations, the unique features of this cultivar have proven to be stable and true to type.

### SUMMARY OF THE INVENTION

The cultivar ‘Mega Mels’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘Mega Mels’. These characteristics in combination distinguish ‘Mega Mels’ as a new and distinct Stokesia laevis cultivar:
1. Stokesia ‘Mega Mels’ exhibits a relatively large plant size; and
2. Stokesia ‘Mega Mels’ exhibits a globular plant shape; and
3. Stokesia ‘Mega Mels’ exhibits large, light purple flowers which are conspicuously centered yellow.

### BRIEF DESCRIPTION OF THE FIGURES

FIG. 1 illustrates, as nearly true as it is reasonably possible to make the same in color photographs of this type, exemplary saleable ‘Mega Mels’ plants grown outdoors in 140 mm nursery containers at the inventor's commercial greenhouse in Rijsbergen, the Netherlands.

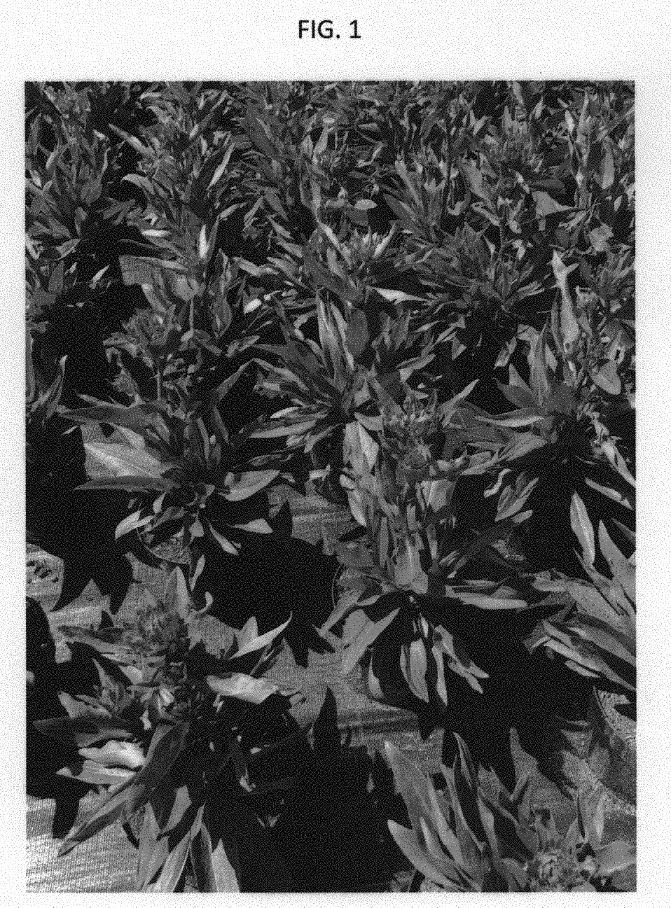

FIG. 2 illustrates, as nearly true as it is reasonably possible to make the same in color photographs of this type, the typical flowers of ‘Mega Mels’.

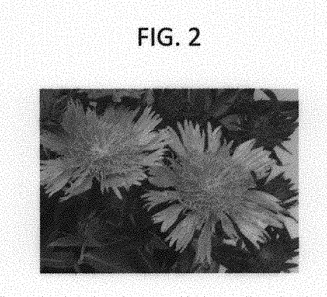

### BOTANICAL DESCRIPTION OF THE PLANT

A detailed botanical description of a new and distinct variety of Stokesia plant known as ‘Mega Mels’ is not yet available. A comparison with the parents and most similar variety of common knowledge are provided below.
General plant description: Botanical description is not yet available.
Root system: Botanical description is not yet available.
Stem: Botanical description is not yet available.
Foliage: Botanical description is not yet available.
Inflorescence: Botanical description is not yet available.
Flower buds: Botanical description is not yet available.
Flower: Botanical description is not yet available.
Reproductive organs: Botanical description is not yet available.
Seed and fruit: Botanical description is not yet available.

### COMPARISONS WITH THE PARENTS

Plants of the new cultivar ‘Mega Mels’ differ from its parent, Stokesia laevis ‘Mels Blue’ (U.S. Plant Pat. No. 23,090), by the characteristics described in Table 1.

TABLE 1

| Characteristic | ‘Mega Mels’ | ‘Mels Blue’ |
|---|---|---|
| Plant height. | Taller than | Shorter than |
| | ‘Mels Blue’. | ‘Mega Mels’. |
| General coloration | Light purple and | Dark purple. |
| of the flower. | lightly suffused | Flowers are |
| | with pink. | inconspicuously |
| | Flowers are | centered yellow. |
| | conspicuously | |
| | centered yellow. | |

Plants of the new cultivar ‘Mega Mels’ differ from its pollen parent, an unnamed Stokesia laevis plant (not patented), by the characteristics described in Chart 2.

CHART 2

| Characteristic | ‘Mega Mels’ | The pollen parent |
|---|---|---|
| Abundance of | More abundant. | Less abundant. |
| foliage. | | |
| Foliage thickness. | Thicker than the | Thinner than |
| | pollen parent. | ‘Mega Mels’. |
| General | Lighter green. | Darker green. |
| coloration of | | |
| mature foliage. | | |
| Floriferousness. | More flowers | Fewer flowers |
| | per plant. | per plant. |
| Abundance of | More abundant. | Less abundant. |
| involucral bracts. | | |

### COMPARISONS WITH THE MOST SIMILAR VARIETY OF COMMON KNOWLEDGE

Plants of the new cultivar ‘Mega Mels’ may be distinguished from the variety Stokesia laevis ‘Mini Mels’ (an application for which is being filed concurrently with the instant plant variety) by the combination of characteristics described in Table 2.

TABLE 2

| Characteristic | ‘Mega Mels’ | ‘Mini Mels’ |
|---|---|---|
| Floriferousness. | Fewer flowers | More flowers |
| | per plant. | per plant. |
| Flower size. | Larger than | Smaller than |
| | ‘Mini Mels’. | ‘Mega Mels’. |
| Abundance of | More abundant. | Less abundant. |
| involucral bracts. | | |
| Attitude of | More relaxed. | More upright. |
| involucral bracts. | | |
| General coloration | Light purple and | Dark purple; |
| of the flower. | lightly suffused | conspicuously |
| | with pink; | centered lighter |
| | inconspicuously | yellow. |
| | centered yellow. | |

That which is claimed is:

1. A new and distinct cultivar of Stokesia plant named ‘Mega Mels’, substantially as described and illustrated herein.
